(19) United States
(12) Patent Application Publication
(10) Pub. No.: US 2009/0221595 A1
(43) Pub. Date: Sep. 3, 2009

(54) Crystalline form of sitagliptin

(76) Inventors: Nurit Perlman, Kfar Saba (IL); Revital Ramaty, Ramat-Hasharon (IL); Eli Lancry, Modiin (IL); Marina Kalujny, Ariel (IL)

Correspondence Address: KENYON & KENYON LLP, ONE BROADWAY, NEW YORK, NY, 10004, US

(21) Appl. No.: 12/313,975

(22) Filed: Nov. 25, 2008

Related U.S. Application Data
(60) Provisional application No. 61/135,005, filed on Jul. 15, 2008.
(60) Provisional application No. 61/134,878, filed on Jul. 14, 2008.
(60) Provisional application No. 61/004,383, filed on Nov. 26, 2007.

(51) Int. Cl. A61K 31/4985; C07D 471/04; A61P 3/10

(52) U.S. Cl. 514/249; 544/350

(57) ABSTRACT

A Sitagliptin crystalline form characterized by PXRD pattern having any 5 peaks selected from the group consisting of 7.4, 11.5, 16.7, 17.7, 18.9, 24.1, 24.5, 27.0, 28.5 and 28.8±0.2 degrees 2-theta, wherein any combination of peaks selected includes the peak at 7.4±0.2 degrees two theta, processes for preparing said Sitagliptin crystalline form, and pharmaceutical compositions thereof, are provided.

### RELATED APPLICATIONS

This application claims the benefit of U.S. provisional application Nos. 61/135,005, filed Jul. 15, 2008, 61/134,878, filed Jul. 14, 2008, and 61/004,383, filed Nov. 26, 2007, hereby incorporated by reference in their entirety.

### FIELD OF THE INVENTION

The invention encompasses a polymorph of sitagliptin, processes for preparing the polymorph, and pharmaceutical compositions thereof.

### BACKGROUND OF THE INVENTION

Sitagliptin, (3R)-3-amino-1-[9-(trifluoromethyl)-1,4,7,8-tetrazabicyclo[4.3.0]nona-6,8-dien-4-yl]-4-(2,4,5-trifluorophenyl)butan-1-one, has the following chemical structure:

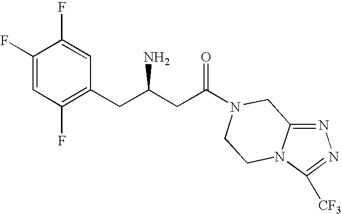

Sitagliptin phosphate is a glucagon-like peptide 1 metabolism modulator, hypoglycemic agent, and dipeptidyl peptidase IV inhibitor. The phosphate form of Sitagliptin is currently marketed in the United States under the tradename JANUVIA™ in its monohydrate form. JANUVIA™ is indicated to improve glycemic control in patients with type 2 diabetes mellitus.

The following PCT Publications describe the synthesis of sitagliptin via stereoselective reduction: WO 2004/087650, WO 2004/085661, and WO 2004/085378.

PCT application No. WO 2004/085661 describes sitagliptin, in its free base form, as a crystalline material, and further reports that the crystalline material tends to melt in the range of 114.1° to 115.7° C.

Polymorphism, the occurrence of different crystal forms, is a property of some molecules and molecular complexes. A single molecule, like sitagliptin, may give rise to a variety of crystalline forms having distinct crystal structures and physical properties like melting point, x-ray diffraction pattern, infrared absorption fingerprint, and solid state NMR spectrum. One crystalline form may give rise to thermal behavior different from that of another crystalline form. Thermal behavior can be measured in the laboratory by such techniques as capillary melting point, thermogravimetric analysis (“TGA”), and differential scanning calorimetry (“DSC”), which have been used to distinguish polymorphic forms.

The difference in the physical properties of different crystalline forms results from the orientation and intermolecular interactions of adjacent molecules or complexes in the bulk solid. Accordingly, polymorphs are distinct solids sharing the same molecular formula yet having distinct advantageous physical properties compared to other crystalline forms of the same compound or complex.

One of the most important physical properties of pharmaceutical compounds is their solubility in aqueous solution, particularly their solubility in the gastric juices of a patient. For example, where absorption through the gastrointestinal tract is slow, it is often desirable for a drug that is unstable to conditions in the patient's stomach or intestine to dissolve slowly so that it does not accumulate in a deleterious environment. Different crystalline forms or polymorphs of the same pharmaceutical compounds can and reportedly do have different aqueous solubilities.

The discovery of new polymorphic forms and solvates of a pharmaceutically useful compound provides a new opportunity to improve the performance characteristics of a pharmaceutical product. It enlarges the repertoire of materials that a formulation scientist has available for designing, for example, a pharmaceutical dosage form of a drug with a targeted release profile or other desired characteristic. Therefore, there is a need for additional crystalline forms of sitagliptin.

### SUMMARY OF THE INVENTION

The present invention encompasses sitagliptin crystalline form characterized by PXRD pattern having any 5 peaks selected from the group consisting of 7.4, 11.5, 16.7, 17.7, 18.9, 24.1, 24.5, 27.0, 28.5 and 28.8±0.2 degrees 2-theta, wherein any combination of peaks selected includes the peak at 7.4±0.2 degrees two theta, and process of preparing the crystalline form.

### BRIEF DESCRIPTION OF THE FIGURES

FIG. 1 illustrates a powder XRD pattern of Form I of sitagliptin.

### DETAILED DESCRIPTION OF THE INVENTION

As used herein, the term “room temperature” refers to a temperature of about 20° C. to about 35° C., more preferably about 25° C. to about 35° C., more preferably about 25° C. to about 30° C., and most preferably about 25° C.

In one embodiment, the present invention encompasses a sitagliptin crystalline form, herein defined as Form I, characterized by a PXRD pattern having any 5 peaks selected from the group consisting of 7.4, 11.5, 16.7, 17.7, 18.9, 24.1, 24.5, 27.0, 28.5 and 28.8±0.2 degrees 2-theta, wherein any combination of peaks selected includes the peak at 7.4±0.2 degrees two theta.

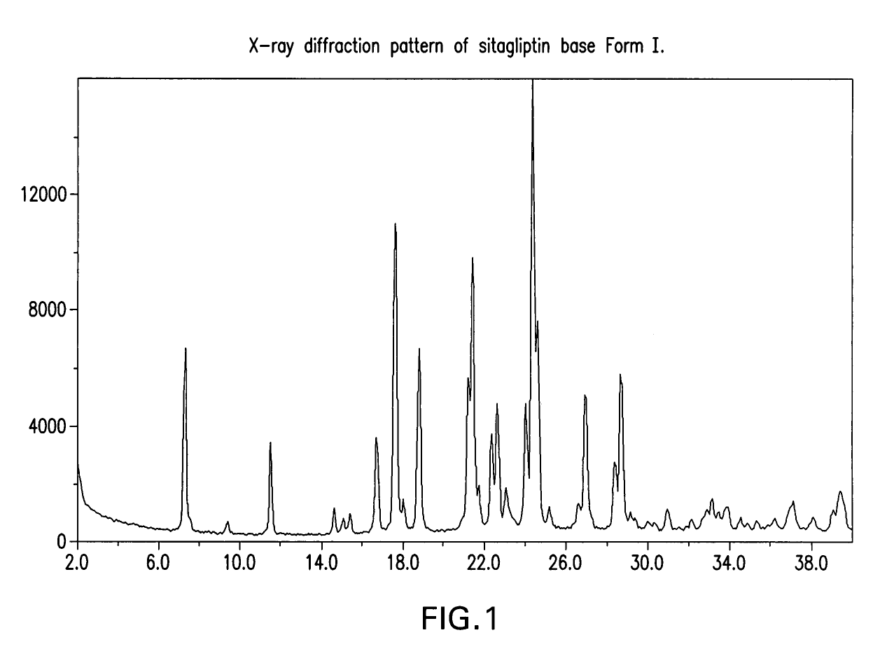

In another embodiment, the present invention encompasses sitagliptin crystalline form I, characterized by a powder XRD pattern as depicted in FIG. 1.

The present invention encompasses sitagliptin crystalline form I, further characterized by data selected from the group consisting of a powder XRD pattern with peaks at about 7.4, 16.7, 17.7, 28.5 and 28.8±0.2 degrees 2-theta; a powder XRD pattern with peaks at about 7.4, 11.5, 16.7, 17.7 and 18.9±0.2 degrees 2-theta; a powder XRD pattern with peaks at about 7.4, 11.5, 16.7, 28.5 and 28.8±0.2 degrees 2-theta and a powder XRD pattern with peaks at about 7.4, 24.1, 24.5, 27.0, and 28.8 degrees 2-theta.

In another embodiment, the present invention encompasses a process for preparing sitagliptin crystalline form I of the present invention, comprising providing a mixture of sitagliptin salt, water, and an inorganic base, and recovering the sitagliptin crystalline form from the reaction mixture. Preferably, from about 2 to about 10 ml of water are used per gram of the sitagliptin salt. Preferably, the sitagliptin salt is sitagliptin phosphate.

Examples for the inorganic base are NaOH, KOH, Na₂CO₃, and K₂CO₃.

In one specific embodiment, sitagliptin salt is slurried in water and further combined with ammonia, to create a two phase system. The sitagliptin crystalline form is further recovered from the organic phase.

Preferably, ammonia is added to the reaction mixture until a pH of about 8 to about 14 is achieved, preferably, about 9 to about 11, and more preferably, about 10.

The crystalline form may be recovered from the reaction mixture by any conventional method. Preferably, the crystalline form is recovered by washing and filtrating the organic phase. Preferably, the obtained sitagliptin base form I is further dried at elevated temperature, preferably under reduced pressure (for example less than 1 atmosphere, more preferably, about 10 mbar to about 100 mbar, more preferably, about 10 mbar to about 25 mbar). Preferably, the drying is carried out at a temperature between about 40° C. and about 60° C., preferably between about 45° C. and about 55° C., most preferably about 50° C. Preferably the drying takes place over a period of about 8 hours to about 36 hours, preferably about 10 hours to about 24 hours, most preferably about 12 hours.

In another embodiment, the present invention encompasses another process for preparing Sitagliptin crystalline form I of the present invention, comprising providing a solution of Sitagliptin base with an organic solvent selected from the group consisting of tetrahydrofuran, dioxane, cyclopentyl methyl ether, C₃-C₆ ester, such as dimethyl carbonate, isopropyl acetate, a C₂-C₄ alcohol, such as ethanol, isopropanol, and 1-propanol, and combinations thereof; cooling the solution; and recovering the sitagliptin crystalline form I from the reaction mixture.

Preferably, from about 1 ml to about 20 ml of the organic solvent is used per 1 gram of the sitagliptin base.

Preferably, the Sitagliptin base is dissolved in the organic solvent at a temperature of about 50° C. to about 100° C., and, more preferably, about 55° C. to about 90° C. Preferably, the solution may be maintained at this temperature for about 1 hour to about 4 hours, and, more preferably, about 2 hours to about 3 hours.

Preferably, the solution is cooled to a temperature of about 0° C. to about 35° C., more preferably, about 20° C. to about 35° C., even more preferably, about 25° C. to about 30° C., and, most preferably, about 25° C. After cooling, the solution is maintained at the temperature for about 8 hours to about 24 hours, preferably, about 10 hours to about 16 hours, and, most preferably, about 12 hours.

Preferably, the obtained sitagliptin base form I is further dried at elevated temperature, and, preferably, under reduced pressure (for example less than 1 atmosphere, more preferably, more preferably, about 10 mbar to about 100 mbar, and, most preferably, about 10 mbar to about 25 mbar). Preferably, the drying is carried out at a temperature between about 40° C. and about 60° C., more preferably, between about 45° C. and about 55° C., and, most preferably, about 50° C. Preferably, the drying takes place over a period of about 8 hours to about 36 hours, more preferably, about 10 hours to about 24 hours, and, most preferably, about 12 hours.

In another embodiment, the present invention encompasses another process for preparing Sitagliptin crystalline form I of the present invention, comprising providing a solution of Sitagliptin base with an organic solvent selected from the group consisting of dioxane, methyl ethyl ketone, propylene glycol monomethyl ether, and methyl isobutyl ketone; adding an antisolvent such as C₅-C₁₀ hydrocarbons, such as cyclohexane, and n-hexane, or water; and recovering the sitagliptin crystalline form I from the reaction mixture. Preferably, from about 1.5 ml to about 2.5 ml of the organic solvent are used per gram of the sitagliptin base. Preferably, from about 20 ml to about 30 ml of antisolvent are used per gram of the sitagliptin base.

Preferably, the Sitagliptin base is dissolved in the organic solvent at a temperature of about 50° C. to about 85° C., and, more preferably, about 55° C. to about 75° C. The solution is preferably maintained at this temperature for about 1 hour to about 4 hours, more preferably 2 hours to about 3 hours.

Preferably the solution is then cooled to a temperature of about 20° C. to about 35° C., more preferably about 25° C. to about 30° C., most preferably about 25° C. The solution is preferably maintained at this temperature for about 8 hours to about 24 hours, preferably about 10 hours to about 16 hours, most preferably about 12 hours.

The antisolvent may be added in one step. The antisolvent may also be added incrementally. For example, in two or more steps, three or more steps, or four or more steps. The steps may be separated by about 1 hour to about 36 hours, 3 hours to about 36 hours, about 8 hours to about 30 hours, preferably, about 10 hours to about 24 hours, and, most preferably, about 12 hours.

Preferably, the obtained Sitagliptin base form I is further dried at elevated temperature, preferably, under reduced pressure (less than 1 atmosphere, more preferably, about 10 mbar to about 100 mbar, and, most preferably, about 10 mbar to about 25 mbar). Preferably, the drying is carried out at a temperature between about 40° C. and about 60° C., more preferably, between about 45° C. and about 55° C., and, most preferably, about 50° C. Preferably the drying takes place over a period of about 8 hours to about 36 hours, more preferably, about 10 hours to about 24 hours, and, most preferably, about 12 hours.

In another embodiment, the present invention encompasses another process for preparing Sitagliptin crystalline form I of the present invention, comprising providing a solution of Sitagliptin base with an organic solvent, selected from the group consisting of ethanol, dimethylformamide, methyl ethyl ketone, and methyl isobutyl ketone, and an antisolvent, preferably, selected from the group consisting of C₅-C₁₀ hydrocarbons such as n-hexane, and cyclohexane, or water; preferably, evaporating the solvents to induce precipitation; and recovering the Sitagliptin crystalline form I from the reaction mixture. Preferably, from about 2 ml to about 8 ml of the organic solvent are used per gram of the sitagliptin base. Preferably, from about 5 ml to about 100 ml of the antisolvent are used per gram of the sitagliptin base.

Preferably, the Sitagliptin base is dissolved in the organic solvent at a temperature of about 50° C. to about 85° C., and, more preferably, about 55° C. to about 75° C. The solution is preferably maintained at this temperature for about 1 hour to about 4 hours, and, more preferably, about 2 hours to about 3 hours.

Preferably the solution is then cooled to a temperature of about 20° C. to about 35° C., more preferably, about 25° C. to about 30° C., and, most preferably, about 25° C. The solution is preferably maintained at this temperature for about 8 hours to about 24 hours, more preferably, about 10 hours to about 16 hours, and, most preferably, about 12 hours.

The antisolvent may be added in one step. The antisolvent may also be added incrementally. For example, in two or more steps, three or more steps, or four or more steps. The steps may be separated by about 1 hour to about 36 hours, 3 hours to about 36 hours, about 8 hours to about 30 hours, preferably about 10 hours to about 24 hours, and, most preferably, about 12 hours.

Preferably, the obtained Sitagliptin base form I is further dried at elevated temperature, preferably under reduced pressure (less than 1 atmosphere, more preferably, about 10 mbar to about 100 mbar, and, most preferably, about 10 mbar to about 25 mbar). Preferably the drying is carried out at a temperature between about 40° C. and about 60° C., more preferably, between about 45° C. and about 55° C., and, most preferably, about 50° C. Preferably the drying takes place over a period of about 8 hours to about 36 hours, more preferably, about 10 hours to about 24 hours, and, most preferably about 12 hours.

In another embodiment, the present invention encompasses another process for preparing sitagliptin crystalline form I, comprising providing a slurry of Sitagliptin base with ethyl acetate and n-hexane; heating the slurry; and, preferably, drying the recovered sitagliptin base at elevated temperature under reduced pressure (less than 1 atmosphere, more preferably, about 10 mbar to about 100 mbar, and, most preferably, about 10 mbar to about 25 mbar) to obtain Sitagliptin base form I.

Preferably the volume ratio of ethyl acetate to n-hexane is about 1:1 to about 1:5, preferably, about 1:2 to about 1:4, and, most preferably, about 1:3.

The slurry is preferably heated to a temperature of about 50° C. to about reflux for about 30 minutes to about 4 hours, and, more preferably, for about an hour. Preferably the solution is then cooled to a temperature of about 20° C. to about 35° C., more preferably, about 25° C. to about 30° C., and, most preferably, about 25° C. The solution is preferably maintained at this temperature for about 8 hours to about 24 hours, more preferably, about 10 hours to about 16 hours, and, most preferably, about 12 hours, before collecting the Sitagliptin base.

Preferably, the obtained sitagliptin base is further dried at elevated temperature, preferably under reduced pressure (for example less than 1 atmosphere, more preferably, about 10 mbar to about 100 mbar, and, most preferably, about 10 mbar to about 25 mbar). Preferably, the drying is carried out at a temperature between about 40° C. and about 60° C., more preferably, between about 45° C. and about 55° C., and, most preferably, about 40° C. Preferably the drying takes place over a period of about 8 hours to about 36 hours, more preferably, about 10 hours to about 24 hours, and, most preferably, about 12 hours.

In another embodiment, the present invention encompasses another process for preparing Sitagliptin crystalline form I comprising providing a solution of Sitagliptin base in trifluoroethanol and methyl tert butyl ether; maintaining the solution for a sufficient period of time to obtain a precipitate; collecting and drying the precipitate at elevated temperature under reduced pressure to obtain Sitagliptin base form I. Preferably, from about 3 ml to about 10 ml of the methyl tert butyl ether are used per gram of the sitagliptin base. Preferably, from about 0.2 ml to about 0.5 ml of the trifluoroethanol are used per gram of the sitagliptin base.

Preferably, the sitagliptin base is prepared by a reduction reaction of (R)-(−)-1-[(S)-2-Diphenylphosphino)ferrocenyl]ethyl di-tert-butylphosphine using a chiral catalyst in the presence of hydrogen and trifluoroethanol. Preferably, the obtained sitagliptin base is further crystallized directly from the reaction mixture.

Preferably, the drying is carried out at elevated temperature, preferably under reduced pressure (for example less than 1 atmosphere, more preferably, about 10 mbar to about 100 mbar, and, most preferably, about 10 mbar to about 25 mbar). Preferably, the drying is carried out at a temperature between about 40° C. and about 60° C., more preferably, between about 45° C. and about 55° C., and, most preferably, about 50° C. Preferably the drying takes place over a period of about 8 hours to about 36 hours, more preferably, about 10 hours to about 24 hours, and, most preferably, about 12 hours.

Preferably, the solution is maintained at about 5° C. to about 50° C., more preferably, at about 15° C. to about 25° C., and, most preferably, at about room temperature for about 4 hours to about 24 hours, and, more preferably, for about 12 hours.

The invention further provides pharmaceutical formulations comprising a crystalline form I of sitagliptin of the present invention. The compositions of the invention include powders, granulates, aggregates and other solid compositions comprising the present invention form of sitagliptin solid crystalline.

The present invention also provides methods of treating type 2 diabetes mellitus in a patient, preferably a human, by administrating to the patient a pharmaceutical composition comprising sitagliptin phosphate crystalline form as described herein. Preferably, the pharmaceutical composition comprises a therapeutically effective amount of sitagliptin phosphate crystalline form.

Having described the invention with reference to certain preferred embodiments, other embodiments will become apparent to one skilled in the art from consideration of the specification. The invention is further defined by reference to the following examples describing in detail the preparation of the composition and methods of use of the invention. It will be apparent to those skilled in the art that many modifications, both to materials and methods, may be practiced without departing from the scope of the invention.

### EXAMPLES

X-Ray powder diffraction data was obtained by using methods known in the art using a SCINTAG powder X-Ray diffractometer model X'TRA equipped with a solid-state detector. Copper radiation of 1.5418 Å was used. A round aluminum sample holder with zero background was used. The scanning parameters included: range: 2-40 degrees two-theta; scan mode: continuous scan; step size: 0.05 deg.; and a rate of 5 deg/min. All peak positions are within ±0.2 degrees two theta.

### Preparation of Form I

### Example 1

An 8 g (0.016 mol) sample of Sitagliptin phosphate was slurried in 176 ml (22 vol.) distilled water and stirred at room temperature (pH˜5-6). NH₃ (26 ml in water; 0.3 mol) was added in portions until pH˜10 was achieved, and the resulting mixture was washed twice with 160 ml (20 vol.) ethyl acetate. The organic phase was dried over Na₂SO₄ and evaporated until a white solid was formed. The solid was dried in a vacuum oven (10-25 mbar) at 50° C. overnight. Yield—87.3%; purity—97.06%.

### Example 2

A sample of STG (sitagliptin) base (100 mg) was dissolved in ethanol (0.5 ml) at 68° C. for 3 hours, then cooled to 25° C., and stirred at 25° C. overnight. Then, 0.5 ml of H₂O was added, and the mixture stirred at 25° C. for over night. Then, 3 ml of H₂O was added in portions, and stirred at 25° C. overnight. Then, 2 ml of n-Hexane and 3 Ml H₂O at 25° C. were added, and the mixture was stirred at 25° C. Crystallization did not occur, and, therefore most of solvent was evaporated. The oil was kept at room temperature for a week. Crystallization occurred. The wet product obtained was STG base crystalline form base I. The sample was dried at 50° C. overnight to obtain a STG base crystalline form base I.

### Example 3

A sample of STG base (100 mg) was dissolved in propylene glycol monomethyl ether (0.5 ml) at 68° C. for 3 hours, then cooled to 25° C., and stirred at 25° C. overnight. Then 1 ml of cyclohexane at 25° C. was added, and the mixture was stirred at 25° C. overnight. Then 2 ml of cyclohexane at 25° C. was added, and the mixture was stirred at 25° C. for 3 hours. The product was isolated by vacuum filtration. The sample was dried at 50° C. overnight to obtain STG base crystalline form base I.

### Example 4

A sample of STG base (100 mg) was dissolved in dimethyl formamide (0.3 ml) at 68° C. for 3 hours. Then the solution was cooled to 25° C., and stirred at 25° C. overnight. Then cyclohexane and H₂O at 25° C. were added, and the mixture was stirred at 25° C. overnight. Then 3 ml of cyclohexane at 25° C. were added, and the mixture was stirred at 25° C. overnight. Then 4 ml of cyclohexane at 25° C. were added, and the mixture was stirred at 25° C. overnight. Most of the solvent was evaporated, and then crystallization occurred. The product was isolated by vacuum filtration to obtain wet STG base crystalline form base I. The sample was dried at 50° C. overnight to obtain STG base crystalline form base I.

### Example 5

A sample of STG base (100 mg) was dissolved in methyl isobutyl ketone (0.8 ml) at 68° C. for 3 hours, cooled to 25° C., and stirred at 25° C. overnight. Then, 1 ml of cyclohexane at 25° C. was added, and the mixture was stirred at 25° C. overnight. Then, 2 ml of cyclohexane at 25° C. were added, and the mixture was stirred at 25° C. overnight. Then, 5 ml of cyclohexane at 25° C. were added, and the mixture was stirred at 25° C. overnight. The solvent was evaporated from a small amount of product, and the product was isolated by vacuum filtration to obtain wet STG base form base I. The sample was dried at 50° C. overnight to obtain STG base crystalline form base I.

### Example 6

A sample of STG base (100 mg) was dissolved in tetrahydrofuran (0.3 ml) at 73° C. for 2.25 hours (2 hours and 15 minutes), then cooled to 25° C., and stirred at 25° C. overnight. The product was isolated by vacuum filtration to obtain wet STG base form base I. The sample was dried at 50° C. overnight to obtain STG base crystalline form base I.

### Example 7

A sample of STG base (100 mg) was dissolved in dimethyl carbonate (0.5 ml) at 73° C. for 2.25 (2 hours and 15 minutes) hours, then cooled to 25° C., and stirred at 25° C. overnight. The product was isolated by vacuum filtration to obtain wet STG base form base I. The sample was dried at 50° C. overnight to obtain STG base crystalline form base I.

### Example 8

A sample of STG base (100 mg) was dissolved in dioxane (0.5 ml) at 73° C. for 2.25 (2 hours and 15 minutes) hours, then cooled to 25° C., and stirred at 25° C. overnight. Then, 0.5 ml of n-hexane at 25° C. was added, and the mixture was stirred at 25° C. for 20 minutes. The product was isolated by vacuum filtration to obtain wet STG base form base I. The sample was dried at 50° C. overnight to obtain STG base crystalline form base I.

### Example 9

A sample of STG base (100 mg) was dissolved in ethanol (0.1 ml) at 55° C. for 2 hours, then cooled to 25° C., and stirred at 25° C. overnight. The product was isolated by vacuum filtration to obtain wet STG base form base I. The sample was dried at 50° C. overnight to obtain STG base crystalline form base I.

### Example 10

A sample of STG base (100 mg) was dissolved in methyl ethyl ketone (0.2 ml) at 55° C. for 2 hours, then cooled to 25° C., and stirred at 25° C. overnight. Then, 2.5 ml of n-hexane at 25° C. were added, and the mixture was stirred at 25° C. overnight. The product was isolated by vacuum filtration to obtain wet STG base form base I (low crystallinity). The sample was dried at 50° C. overnight to obtain STG base crystalline form base I.

### Example 11

A sample of STG base (100 mg) was dissolved in propylene glycol monomethyl ether (0.2 ml) at 55° C. for 2 hours, then cooled to 25° C., and stirred at 25° C. overnight. Then, 0.5 ml of n-hexane at 25° C. was added, and the mixture was stirred at 25° C. overnight. The product was isolated by vacuum filtration to obtain wet STG base form base I. The sample was dried at 50° C. overnight to obtain STG base crystalline form base I.

### Example 12

A sample of STG base (100 mg) was dissolved in methyl isobutyl ketone (0.2 ml) at 55° C. for 2 hours, then cooled to 25° C., and stirred at 25° C. overnight. Then, 2.5 ml of n-hexane at 25° C. was added, and the mixture was stirred at 25° C. overnight. The product was isolated by vacuum filtration to obtain wet STG base form base I (low crystallinity). The sample was dried at 50° C. overnight to obtain STG base crystalline form base I.

### Example 13

A sample of STG base (100 mg) was dissolved in dioxane (0.2 ml) at 55° C. for 2 hours, then cooled to 25° C., and stirred at 25° C. overnight. The product was isolated by vacuum filtration to obtain wet STG base form base I. The sample was dried at 50° C. overnight to obtain STG base crystalline form base I.

### Example 14

A sample of STG base (100 mg) was slurried in trifluorotoluene (0.5 ml) at 70° C. for 2 hours, and then cooled to 25° C., and stirred at 25° C. overnight. The product was isolated by vacuum filtration to obtain wet STG base form base I.

### Example 15

A sample of wet STG base (1 g) was dissolved in cyclopentyl methyl ether (20 ml) at 90° C. The solution was cooled to room temperature, then cooled with an ice bath, and stirred overnight. The product was isolated by vacuum filtration and dried at 50° C. in a vacuum oven (10-25 mbar) for 24 hours to obtain STG base form I (850 mg).

### Example 16

A sample of wet STG base (1 g) was dissolved in isopropanol (IPA) (8 ml) at 80° C. The solution was cooled to room temperature and stirred overnight. The product was isolated by vacuum filtration, and dried at 50° C. in a vacuum oven (10-25 mbar) for 24 hours to obtain STG base form I (540 mg).

### Example 17

A sample of wet STG base (1 g) was dissolved in 1-Propanol (9 ml) at 80° C. The solution was cooled to room temperature, and stirred overnight. The product was isolated by vacuum filtration, and dried at 50° C. in a vacuum oven (10-25 mbar) for 24 hours to obtain STG base form I (200 mg).

### Example 18

A sample of wet STG base (1 g) was dissolved in isopropyl acetate (12 ml) at 80° C. The solution was cooled to room temperature, then cooled with an ice bath, and stirred overnight. The product was isolated by vacuum filtration, and dried at 50° C. in a vacuum oven (10-25 mbar) for 24 hours to obtain STG base form I (400 mg).

### Example 19

STG base (2.50 g) was slurried in Ethyl acetate: n-hexane 1:3 (75 ml) at reflux for 55 minutes. Then cooled to room temperature and stirred at room temperature for over night.

The product was isolated by vacuum filtration. The sample was dried at 40° C. over night to obtain a STG base crystalline form base I.

### Example 20

To 30 ml of degassed trifluoroethanol (TFE) were added Ruthenium(II) chloride 1,5-cyclooctadiene complex (18.2 mg, 0.037 mmol) and (R)-(−)-1-[(S)-2-Diphenylphosphino)ferrocenyl]ethyl di-tert-butylphosphine (44.8 mg, 0.083 mmol). The solution was degassed again, and left to stir at room temperature for 1 hour. To 250 ml glass reactor were added (Z)-3-amino-1-(3-(trifluoromethyl)-5,6-dihydro-[1,2,4]triazolo[4,3-a]pyrazin-7(8H)-yl)-4-(2,4,5-trifluorophenyl)but-2-en-1-one (30 g, 74.07 mmol) and 100 ml TFE. The slurry was stirred and washed three times with N₂. Than, the catalyst solution was added and the mixture washed three times with N₂, then switched to H₂ and washed three times. The H₂ pressure was set to constant pressure of 5 bar and the reaction was heated to 55° C. for 23 hours. The TFE solution (93.3% purity and 77% R) was evaporated to yield oily-STG-base.

The solution of STG base (ca. 6 g) was concentrated until the percentage of trifluoroethanol was 39%. This solution was dissolved in methyl t-butyl ether (30 ml) at room temperature and stirred at room temperature overnight. The product was isolated by vacuum filtration. The sample was dried at 40° C. over night to obtain a STG base crystalline form base I.

What is claimed is:

1. A process for preparing the Sitagliptin crystalline form I, comprising combining a Sitagliptin salt, water, and an inorganic base to obtain the Sitagliptin crystalline Form I.

2. The process according to claim 1, wherein the Sitagliptin salt is Sitagliptin phosphate.

3. The process according to claim 1, wherein the inorganic base is NaOH, KOH, Na₂CO₃, K₂CO₃, or ammonia.

4. The process according to claims 1, wherein the inorganic base is used in an amount sufficient to achieve a pH of about 8 to about 14.

5. The process according to claim 1, wherein the inorganic base is used in an amount sufficient to achieve a pH of about 9 to about 11.

6. The process according to claim 1, wherein the inorganic base is used in an amount sufficient to achieve a pH of about 10.

7. A process for preparing the Sitagliptin crystalline form I, comprising:
preparing a solution of a Sitagliptin base in an organic solvent selected from the group consisting of tetrahydrofuran, dimethyl carbonate, ethanol, dioxane, cyclopentyl methyl ether, isopropanol, 1-propanol, isopropyl acetate, and combinations thereof; and
cooling the solution to obtain the Sitagliptin crystalline Form I.

8. The process according to claim 7, wherein the organic solvent is a C₂ to C₄ alcohol.

9. The process according to claim 8, wherein the C₂ to C₄ alcohol is selected from the group consisting of ethanol, isopropanol, and 1-propanol.

10. The process according to claim 7, wherein the organic solvent is selected from the group consisting of tetrahydrofuran, dimethyl carbonate, dioxane, cyclopentyl methyl ether, and isopropyl acetate.

11. The process according to claim 7, wherein the Sitagliptin base is dissolved in the organic solvent at a temperature of about 50° C. to about 100° C.

12. The process according to claim 7, wherein the Sitagliptin base is dissolved in the organic solvent at a temperature of about 55° C. to about 90° C.

13. The process according to claim 7, wherein the solution is cooled to about 0° C. to about room temperature.

14. The process according to claim 7, wherein the solution is cooled to about 20° C. to about 35° C.

15. The process according to claim 7, wherein the solution is cooled to about 20° C. to about 25° C.

16. The process according to claim 7, wherein the solution is cooled to about 25° C.

17. A process for preparing the Sitagliptin crystalline form I, comprising preparing a solution of a Sitagliptin base with an organic solvent; adding an antisolvent; and maintaining the solution to obtain the Sitagliptin crystalline Form I.

18. The process according to claim 17, wherein the organic solvent is selected from the group consisting of ethanol, dimethylformamide, propylene glycol monomethyl ether, methyl ethyl ketone, and methyl isobutyl ketone.

19. The process according to claim 17, wherein the antisolvent is selected from the group consisting of a C₅-C₁₀ saturated hydrocarbon and water.

20. The process according to claim 19, wherein the C₅-C₁₀ saturated hydrocarbon is n-hexane or cyclohexane.

21. The process according to claim 17, wherein the organic solvent is selected from the group consisting of dioxane, methyl ethyl ketone, propylene glycol monomethyl ether, and methyl isobutyl ketone; and wherein the antisolvent is n-hexane.

22. The process according to claim 17, wherein the Sitagliptin base is dissolved in the organic solvent at a temperature of about 50° C. to about 85° C.

23. The process according to claim 17, wherein the Sitagliptin base is dissolved in the organic solvent at a temperature of about 55° C. to about 75° C.

24. The process according to claim 17, further comprising evaporating the solvent and the antisolvent to induce precipitation.

25. A process for preparing the Sitagliptin crystalline form I, comprising:
preparing a slurry of a Sitagliptin base with a mixture of an organic solvent and an antisolvent; and
maintaining the solution to obtain the Sitagliptin crystalline Form I.

26. The process according to claim 25, wherein the organic solvent is ethyl acetate.

27. The process according to claim 25, wherein the antisolvent is selected from the group consisting of a C₅-C₁₀ saturated hydrocarbon and water.

28. The process according to claim 27, wherein the C₅-C₁₀ saturated hydrocarbon is n-hexane.

29. The process according to claim 25, wherein the volume ratio of the organic solvent and the antisolvent is about 1:3.

30. The process according to claim 25, wherein the Sitagliptin base is admixed in the organic solvent at a temperature of about 50° C. to about reflux.

31. The process according to claim 25, further comprising drying the precipitate at elevated temperature under reduced pressure.

32. A process for preparing the Sitagliptin crystalline form I, comprising:
preparing a solution of a Sitagliptin base with trifluoroethanol and methyl tert butyl ether;
recovering the precipitate from the solution; and
drying the precipitate at elevated temperature under reduced pressure to obtain the Sitagliptin crystalline Form I.

33. A process for preparing the Sitagliptin crystalline form I, comprising:
preparing a solution of a Sitagliptin base with an organic solvent; adding an antisolvent;
and evaporating the solvent and the antisolvents to obtain the Sitagliptin crystalline Form I.

34. The process according to claim 33, wherein the solvent is selected from the group consisting of ethanol, dimethylformamide, methyl ethyl ketone, and methyl isobutyl ketone.

35. The process according to claim 33, wherein the antisolvent is selected from the group consisting of n-hexane, cyclohexane, and water.

36. A solid containing a pharmaceutical composition comprising the Sitagliptin crystalline form I and at least one pharmaceutically suitable excipient.
